(19) United States
(12) Patent Application Publication
(10) Pub. No.: US 2022/0133490 A1
(43) Pub. Date: May 5, 2022

(54) Methods and Implants for Facet Joint Stabilization or Fusion

(71) Applicant: Michael Murray, Wayne, PA (US)

(72) Inventor: Michael Murray, Wayne, PA (US)

(21) Appl. No.: 17/518,807

(22) Filed: Nov. 4, 2021

Related U.S. Application Data
(60) Provisional application No. 63/109,904, filed on Nov. 5, 2020.

(51) Int. Cl. A61F 2/44; A61F 2/46

(52) U.S. Cl. CPC A61F 2/4405; A61F 2002/30785; A61F 2/4611; A61F 2/447

(57) ABSTRACT

A method for facet joint stabilization or fusion includes inserting an implant into a facet joint of a patient and introducing a bone cement into the facet joint. The implant includes a body that is sized and configured to be entirely contained in the facet joint. The bone cement may be introduced through a first channel of the body of the implant. A portion of the bone cement may flow back into the implant through a second channel in the body of the implant.

### CROSS-REFERENCE TO RELATED APPLICATIONS

This application claims priority to and the benefit of U.S. Provisional Patent Application No. 63/109,904, filed Nov. 5, 2020, which is incorporated herein by reference in its entirety.

### FIELD

The present disclosure, according to some embodiments, provides methods and implants that are useful for stabilizing or fusing a facet joint. In further embodiments, the present disclosure provides tools and kits that are useful carrying out the described methods.

### BACKGROUND

In the posterior aspect of the lumbar spine, the anatomic structures that are responsible for motion are the facet joints. Facet joints are a set of synovial, plane joints between the articular processes of two adjacent vertebrae. FIG. 1 illustrates a portion of a human spine showing the location of facet joints (labeled as “10”) between adjacent vertebrae.

Currently, the standard of care for posterior stabilization of the lumbar spine is pedicle screw fixation. Pedicle screws are designed to prevent motion in the facet joints by anchoring into the bony pedicles that are located above and below the facet joint in question. The heads of the screws are then fastened to a rigid rod that connects the screws and thereby prevents motion of the facet joint. However, the placement of pedicle screws is disruptive to the surrounding muscles, carries the potential risk of nerve damage during their insertion, and can cause temporary and/or persistent post-operative pain due to their prominence. For example, FIG. 2A illustrates correct positioning of pedicle screws 12, whereas FIG. 2B illustrates a misplaced pedicle screw impinging on neural structures of the spine.

### SUMMARY

In some embodiments, the present disclosure provides a facet joint stabilization and fusion device that can avoid or overcome the problems discussed above. In one embodiment, there is an intra-facet stabilization implant that includes is a rigid device placeable into the facet joint. The rigid device is placeable to prevent motion via the exemplary mechanism of friction (e.g., between the device and the articulating surfaces of the facet joint). In one embodiment, the intra-facet stabilization devices include adequate surface area contact between the stabilization device and the nearly infinite variations in the anatomy of a facet joint. In one embodiment, the intra-facet stabilization devices are configured to accommodate fixation of facet joints in the spine that are oriented in a sagittal manner that would avoid unfavorable shear forces being placed upon the implant-facet joint interface.

In some embodiments, a facet joint stabilization and fusion device of the present disclosure incorporates a rigid implant that is sized and configured to be fully positioned within a facet joint such that no portion of the implant protrudes beyond the facet joint. In some embodiments, the rigid implant is configured to be utilized with a bone cement. The bone cement may include, for example, polymethyl methacrylate (PMMA) or other acrylic bone cements, calcium phosphate cements (CPCs), hydroxyapatite bone cements (HBCs), calcium sulfate cements (CSCs), and combinations thereof (e.g., calcium phosphate/hydroxyapatite or PMMA/calcium phosphate composite materials, etc.). In some embodiments, the bone cement may include any known bone cement that is useful for kyphoplasty or vertebroplasty. In some embodiments, the bone cement is PMMA or other material that mimics the properties of PMMA. The bone cement may be introduced in a fluid or flowable condition (e.g., injectable) and allowed to cure or harden after introduction into the facet joint. In some embodiments, the rigidity of the implant combined with the fluidity of the bone cement allows for an optimal surface of contact area with the facet joint. In some embodiments, this configuration will maximize the biomechanical strength of the facet joint stabilization. In addition, the ability to fill each aspect of the facet joint with bone cement, will maximize the amount of the construct that is under compressive load (the most tolerated form of force) as opposed to shear force.

In some embodiments, an implant for insertion into a facet joint includes a body that is sized to fit completely within the facet joint. In some embodiments, the body of the implant is constructed from a metal or metal alloy (e.g., titanium or titanium alloy). In some embodiments, the body includes a proximal end, a distal end opposite of the proximal end, the distal end having a tapered portion, and a first channel extending through the body from the proximal end to the distal end. In some embodiments, the body includes a second channel extending through the body from the proximal end to the distal end, the second channel being positioned between the first channel and a first lateral side of the body. In some embodiments, the body includes a third channel extending through the body from the proximal end to the distal end, the third channel being positioned between the first channel and a second lateral side of the body. In some embodiments, a width of the body between the first lateral side and the second lateral side is selected from a range of 5 mm to 8 mm, a length of the body between the proximal end and the distal end is selected from a range of 5 mm to 8 mm, and a height of the body at the proximal end is selected from a range of 2 mm to 4 mm.

In some embodiments, the body further includes a first tool engagement feature positioned on the first lateral side, and a second tool engagement feature positioned on the second lateral side. In some embodiments, the first tool engagement feature and the second tool engagement feature are discrete surface features on the first and second lateral sides. In some embodiments, the first tool engagement feature and the second tool engagement feature include one or more of an indent, recess, groove, slot, notch, protrusion, lip, flange, or the like sized and dimensioned to receive a portion of a tool.

In some embodiments, the body includes a top side having a first planar portion extending from the first lateral side to the second lateral side, and a bottom side opposite the top side and having a second planar portion extending from the first lateral side to the second lateral side. In some embodiments, the first planar portion of the top side and the second planar portion of the bottom side are substantially parallel to each other. In other embodiments, the first planar portion of the top side is acutely angled with respect to the second planar portion of the bottom side. In some such embodiments, the first planar portion of the top side is acutely angled with respect to the second planar portion of the bottom side by an angle selected from 7° to 15°.

In some embodiments, the first channel is disposed about a central axis of the body. In some embodiments, the body includes at least one plane of symmetry that intersects with the central axis. In some embodiments, the body includes two perpendicular planes of symmetry that intersect at the central axis. In some embodiments, the second and third channels are substantially parallel to the first channel. In some embodiments, the first channel, the second channel, and/or the third channel are unthreaded. In some embodiments, the body of the implant includes at least one opening on the first lateral side, the at least one opening on the first lateral side connecting to the second channel. In some embodiments, the body of the implant includes at least one opening on the second lateral side, the at least one opening on the second lateral side connecting to the third channel.

In some embodiments, a method for facet joint stabilization or fusion includes inserting any of the implants described herein into a facet joint of a patient. The facet joint may be a facet joint of the lumbar spine according to some embodiments. In some embodiments, the method further includes introducing a bone cement (e.g., PMMA, CPCs, HBCs, CSCs, etc.) into the facet joint. The bone cement is introduced into the facet joint in an uncured state. In some embodiments, the bone cement is introduced through the first channel of the body of the implant. In some embodiments, a portion of the bone cement exits the first channel and is allowed to at least partially fill a space of the facet joint surrounding the implant. In some embodiments, a portion of the bone cement that exits the first channel is allowed to flow into the second channel and/or the third channel of the body of the implant.

In some embodiments, a method according to the present disclosure includes coupling the implant to an inserter tool having a clamp configured to hold the implant in an insertion position relative to the facet joint. In some embodiments, the clamp comprises a pair of movable arms, and coupling the implant to the inserter tool includes causing the implant to engage the clamp between the pair of movable arms. In some embodiments, the inserter tool includes a hollow shaft that is axially aligned with the first channel when the implant is coupled to the inserter tool.

In some embodiments, the method further includes inserting an impactor tool into the hollow shaft of the inserter tool, the impactor tool having a tip portion that extends into the first channel of the implant when the impactor tool is fully inserted into the hollow shaft of the inserter tool. In some embodiments, the impactor tool is securable to the inserter tool with a threaded coupling. In some embodiments, the method includes removing the impactor tool from the hollow shaft of the inserter tool after inserting the implant into the facet joint of the patient. In some embodiments, after removing the impactor tool from the hollow shaft of the inserter tool, a cement introducer is inserted into the hollow shaft of the inserter tool. In some such embodiments, the cement introducer includes a hollow tip portion that extends into the first channel of the implant when the cement introducer is fully inserted into the hollow shaft of the inserter tool, and a fitting at an end opposite from the hollow tip portion, the fitting configured to couple to a bone cement source.

In some embodiments, the method includes coupling a bone cement source (e.g., syringe or other bone cement dispenser) to the fitting of the cement introducer, and causing bone cement to flow from the bone cement source through the cement introducer and into first channel of the implant. In some embodiments, the method includes causing bone cement to exit from the first channel of the implant to at least partially fills a space of the facet joint surrounding the implant. In some embodiments, the method further includes causing a portion of the bone cement to flow from the space of the facet joint into the second channel and/or third channel in the body of the implant.

### BRIEF DESCRIPTION OF THE DRAWINGS

The foregoing summary, as well as the following detailed description, will be better understood when read in conjunction with the appended drawings. For the purpose of illustrating the present disclosure, there are shown in the drawings exemplary embodiments wherein like reference numerals indicate like elements throughout. It should be noted that aspects of the present disclosure can be embodied in different forms and thus should not be construed as being limited to the illustrated embodiments set forth herein. The elements illustrated in the accompanying drawings are not necessarily drawn to scale, but rather, may have been exaggerated to highlight particular features of the subject matter therein. Furthermore, the drawings may have been simplified by omitting elements that are not necessary for the understanding of the disclosed embodiments.

FIG. 1 is an illustration showing the location of a facet joint between adjacent vertebrae in the lumbar spine of a human.

FIG. 2A-2B are illustrations of the placement of pedicle screws in a human vertebra according to a conventional method of spinal fusion. FIG. 2A shows the correct placement of the pedicle screws. FIG. 2B shows an incorrect placement of a pedicle screw, causing impingement against neural structures.

FIG. 3A illustrates a front, top perspective view of an implant according to certain embodiments of the present disclosure showing the distal end of the implant.

FIG. 3B illustrates a rear, top perspective view of the implant of FIG. 3A showing the proximal end of the implant according to certain embodiments of the present disclosure.

FIG. 3C illustrates a top plan view of the implant of FIG. 3A according to certain embodiments of the present disclosure.

FIG. 3D illustrates a lateral side view of the implant of FIG. 3A according to certain embodiments of the present disclosure.

FIG. 3E illustrates a rear view of the implant of FIG. 3A, showing the proximal end of the implant according to certain embodiments of the present disclosure.

FIG. 4A illustrates a front, top perspective view of an implant according to certain alternative embodiments of the present disclosure, wherein the implant includes tapered top and bottom sides.

FIG. 4B illustrates a lateral side view of the implant of FIG. 4A according to certain embodiments of the present disclosure.

FIG. 5A illustrates a front, top perspective view of the implant of FIG. 3A additionally including one or more lateral openings according to certain embodiments of the present disclosure.

FIG. 5B illustrates a front, top perspective view of the implant of FIG. 4A additionally including one or more lateral openings according to certain embodiments of the present disclosure.

FIG. 5C illustrates a top plan view of the implant of FIG. 5A or 5B according to certain embodiments of the present disclosure.

FIG. 6A illustrates an implant in combination with an inserter tool according to certain embodiments of the present disclosure, wherein the inserter tool is in a disengaged configuration.

FIG. 6B illustrates the implant and inserter tool of FIG. 6A, wherein the inserter tool is in an engaged configuration and coupled to the implant according to certain embodiments of the present disclosure.

FIGS. 7A-7B illustrate an embodiment of an inserter tool according to the present disclosure in an engaged configuration (FIG. 7A) and in a disengaged configuration (FIG. 7B).

FIGS. 8A-8B illustrate a further embodiment of an inserter tool according to the present disclosure in an disengaged configuration (FIG. 8A) and in an disengaged configuration (FIG. 8B).

FIGS. 9A-9C illustrate an impaction tool in combination with an implant and inserter tool according to certain embodiments of the present disclosure.

FIG. 10 illustrates a cement introducer tool in combination with an implant and inserter tool according to certain embodiments of the present disclosure.

FIG. 11 illustrates a bone cement source (e.g., syringe) in combination with an implant, inserter tool, and cement introducer tool according to certain embodiments of the present disclosure.

FIG. 12 illustrates the flow of bone cement through an implant positioned within a facet joint according to certain embodiments of the present disclosure.

FIG. 13 illustrates a set of trial gauges of differing sizes according to certain embodiments of the present disclosure.

FIG. 14 illustrates a set of rasps of differing sizes according to certain embodiments of the present disclosure.

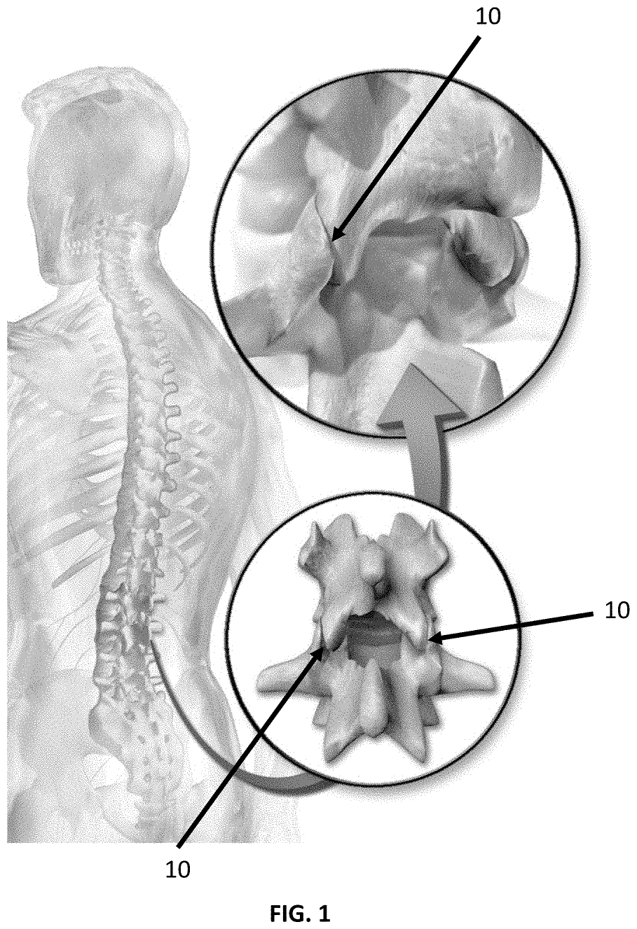

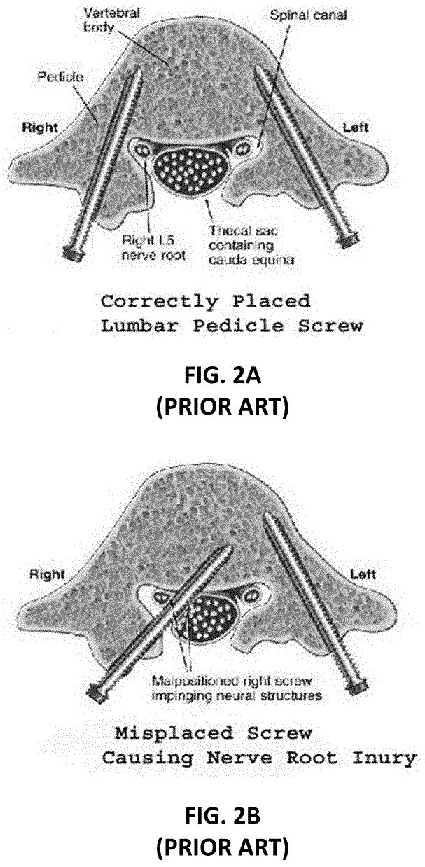

### DETAILED DESCRIPTION

The present subject matter will now be described more fully hereinafter with reference to the accompanying Figures, in which representative embodiments are shown. The present subject matter can, however, be embodied in different forms and should not be construed as limited to the embodiments set forth herein. Rather, these embodiments are provided to describe and enable one of skill in the art.

A method for facet joint stabilization or fusion according to embodiments of the present disclosure includes positioning an implant within a facet joint and introducing a volume of bone cement (e.g., PMMA, CPCs, HBCs, CSCs, etc.) into the facet joint to at least partially surround the implant with the bone cement. In some embodiments, the implant is sized and configured to fit entirely within the facet joint. The facet joint, in some embodiments, may be a facet joint of the lumbar spine. In some embodiments, the bone cement is introduced into the facet joint through the implant itself. In some such embodiments, at least a portion of the volume of the bone cement is passed through a first channel in the implant and exits the implant to fill a space between the implant and a surface of the facet joint. In further embodiments, at least a portion of the bone cement, after exiting the first channel of the implant, is allowed to flow back into the implant through a second channel of the implant.

Methods according to embodiments of the present disclosure may be carried out during open spine surgery or through minimally invasive surgery, e.g., by utilizing a tubular retractor. In open spine surgery, the facet joint may be directly visualized and can be accessed directly by the surgeon. In embodiments using a tubular retractor, a surgeon may use intraoperative fluoroscopy or computerized navigation, for example, to place a tubular retractor over the facet joint that is to be fused or stabilized and be positioned in-line with the opening of the facet joint. Methods according to the present disclosure, may be performed bilaterally, assuming there is no facet joint cyst or compromise of the facet joint at the spinal level in question that would allow for cement extravasation into the spinal canal.

In some embodiments, once the facet joint is identified, electrocautery can be used to remove the dorsal aspect of the facet joint capsule that is overlying the facet joint to expose the opening of the facet joint. In some embodiments, tapered trial gauges of different predetermined dimensions (e.g., 1 mm, 2 mm, 3 mm, 4 mm) may be introduced into the facet joint in order to dilate the opening of the facet joint and to determine the appropriate height of the facet joint implant. In some embodiments, progressively larger trial gauges may be individually inserted into the facet joint until a trial gauge that provides a firm fit within the facet joint is found. In some embodiments, once the appropriate height of the facet joint has been determined, a rasp will be used to remove the cartilage and decorticate the facet joint surfaces. In some embodiments, the height of the rasp that will be used will be selected to be the same as the height of the trial gauge that had the appropriate fit within the facet joint.

In some embodiments, after the facet joint has been rasped, the implant can be secured to an inserter tool. The inserter tool, in some embodiments, includes a hollow shaft configured to abut against an end of the implant and a clamping mechanism configured to engage with one or more sides of the implant to hold the implant. In some embodiments, the clamping mechanism includes two or more movable arms that couple to features on the implant. The features may be, for example, small indents on opposite sides of the implant, according to some embodiments.

In some embodiments, once the inserter tool has been engaged with the implant, an impaction tool will be inserted inside of the hollow shaft of the inserter tool. In some embodiments, the impaction tool includes a tip that will engage with the implant. In some embodiments, the tip of the impaction tool is inserted into a channel in the implant when the impaction tool is seated inside of the inserter tool. In some embodiments, the impaction tool may be secured to the inserter tool via a threaded coupling at an end of the inserter tool. In some embodiments, the impaction tool is configured to stabilize the implant and will facilitate insertion of the implant into the facet joint. In some embodiments, the implant while engaged with the inserter tool and impaction tool, may be driven into the facet joint via light malleting. The malleting may be applied against an end surface of the impaction tool opposite the tip in some embodiments.

In some embodiments, the implant may be inserted into the facet joint until an end of the implant is flush with the opening of the facet joint. In some embodiments, the impaction tool can then be removed from the inserter tool and replaced with a cement introducer tool. In some embodiments, the cement introducer includes a hollow shaft for conducting bone cement from a bone cement source into the facet joint. In some embodiments, the cement introducer is inserted into the hollow shaft of the inserter tool and may also be secured to the inserter tool via a threaded coupling at an end of the inserter tool. In some embodiments, the cement introducer further includes a hollow tip that extends into a channel of the implant for conducting the bone cement into the implant.

In some embodiments, bone cement (e.g., PMMA, CPCs, HBCs, CSCs, etc.) can be introduced through the cement introducer and into the facet joint via a bone cement source coupled to the cement introducer. In some embodiments, the cement introducer includes a fitting for coupling directly or indirectly to the bone cement source. The bone cement source may include any conventional bone cement applicators or dispensers used in kyphoplasty and vertebroplasty. In some embodiments, the bone cement source includes, for example, a syringe containing the bone cement or a cannula and plunger system. In some embodiments, bone cement is channeled from the bone cement source, through the hollow shaft of the cement introducer, and into the facet joint. In some embodiments, the bone cement passes through a first channel in the implant and exits into the facet joint. In some embodiments, as the facet joint fills with bone cement, a portion of the bone cement is allowed to back flow and enter a second and/or third channel within the implant. In some embodiments, some of the bone cement may exit the second and/or third channels through openings located at an end of the implant. In some embodiments, some of the bone cement may exit the second and/or third channels through one or more openings on the lateral sides of the implant. In some embodiments, once bone cement is visualized within these channels, the introduction of bone cement may be ceased. Any bone cement that has extruded out of the facet joint or from the second and/or third channels of the implant may be removed. In some embodiments, after the introduction of the bone cement has ceased, the inserter tool and cement introducer may be removed from the implant. In some embodiments, the bone cement in the facet joint is allowed to fully cure and harden.

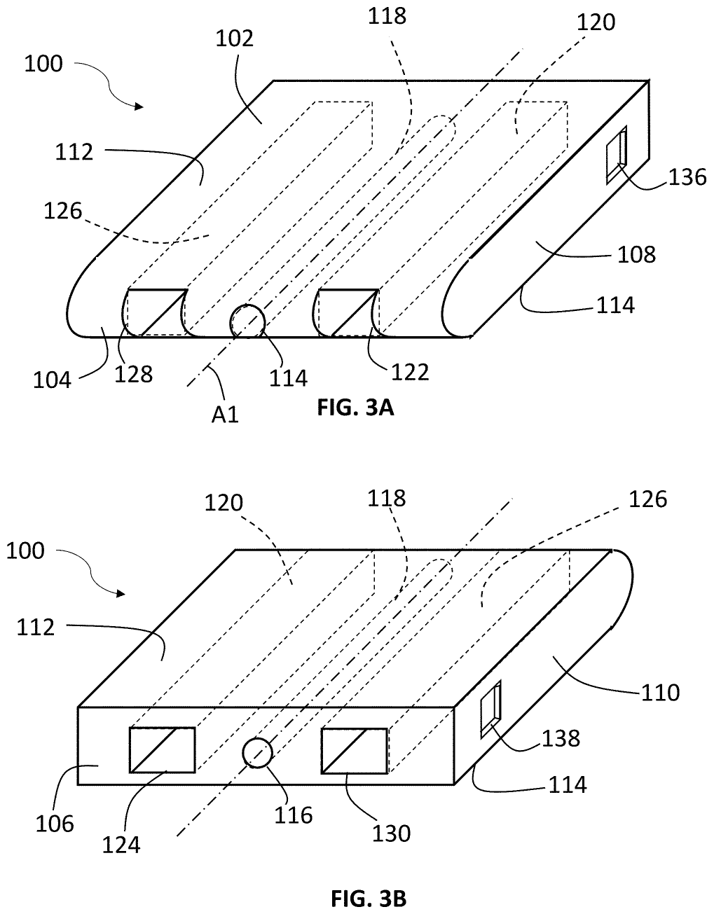

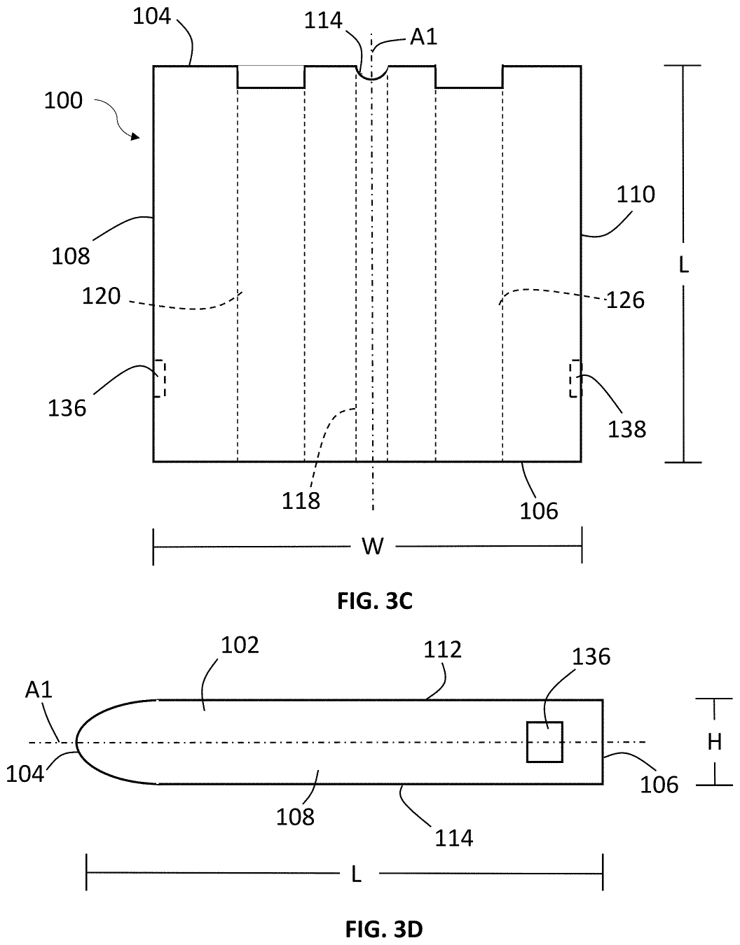

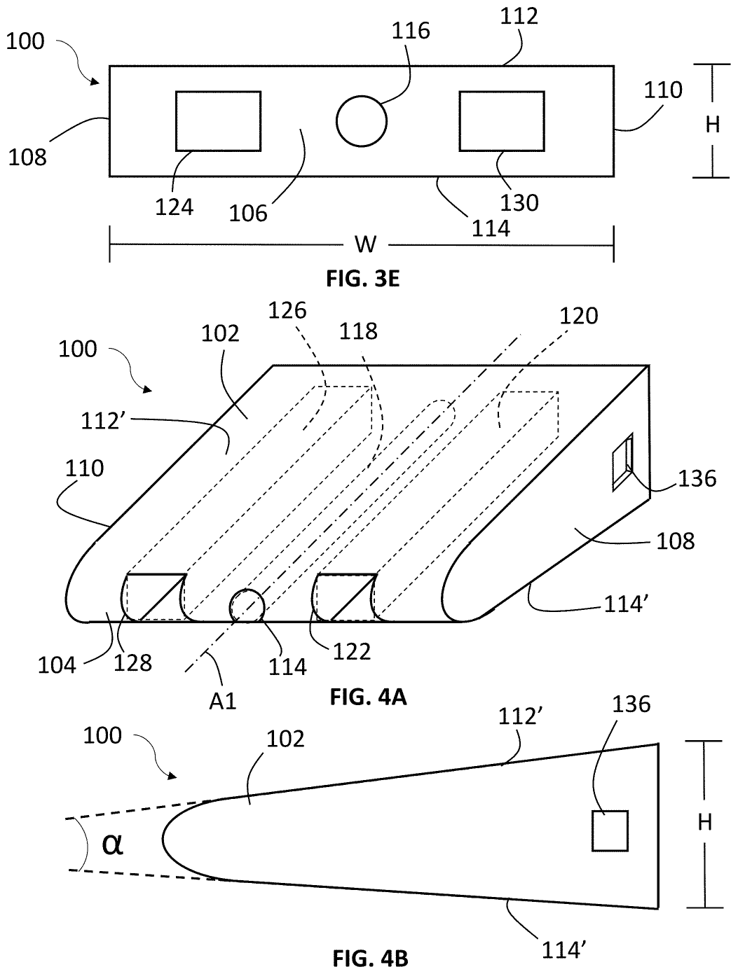

Referring now to the drawings in detail, there is shown in FIGS. 3A-3E an implant 100 for facet joint stabilization and fusion in accordance with certain exemplary embodiments of the present disclosure. In some embodiments, implant 100 is sized and configured to be inserted into and completely contained within a facet joint between two adjacent vertebrae of a subject (e.g., a human patient). In some embodiments, implant 100 includes a body 102 having a distal end 104, a proximal end 106 that is opposite of distal end 104, and lateral sides 108, 110 that extend from proximal end 106 to distal end 104. Body 102 further includes a top side 112 and a bottom side 114 that is opposite of top side 112. The terms “top” and “bottom” are used herein as relative terms and should not be construed as being absolute orientations.

As shown in FIGS. 3C and 3D, implant 100 includes a length L, a width W, and a height H. In some embodiments, length L represents a distance between proximal end 106 and distal end 104, width W represents a distance between lateral sides 108 and 110, and height H represents a distance between top side 112 and bottom side 114. In some embodiments, length L may be selected to be any value from 5 mm to 8 mm, width W may be selected to be any value from 5 mm to 8 mm, and height H may be selected to be any value from 2 mm to 4 mm. The values of width W and height H may be the values measured at proximal end 106. In some embodiments, length L and width W are selected to be the same such that a ratio of length L to width W is equal to 1. In other embodiments, the ratio of length L to width W may be selected to be any value from 0.625 to 1, or from 1 to 1.6. In some embodiments, a ratio of height H to length L may be selected to be any value from 0.25 to 0.4. In some embodiments, a ratio of height H to width W may be selected to be any value from 0.25 to 0.4.

In some embodiments, lateral sides 108 and 110 are substantially planar and parallel such that width W is generally constant along the length of implant 100. In alternative embodiments, lateral sides 108, 110 may, at least partially, taper towards each other at distal end 104 such that width W decreases at distal end 104. In some embodiments, top side 112 and bottom side 114 may be, at least partially, parallel to each other. In some embodiments, top side 112 and bottom side 114 each may include substantially planar portions that are parallel to each other. In some embodiments, at least a portion of top side 112 and bottom side 114 may taper or curve towards each other at distal end 104. In an alternative embodiment illustrated in FIGS. 4A and 4B, top side 112′ and bottom side 114′ are not parallel and may include planar portions that are acutely angled relative to each other. In some embodiments, top side 112′ and bottom side 114′ include planar portions that taper towards each other from proximal end 106 towards distal end 104 at a predetermined angle α. In some embodiments, angle α may be selected to be any value from 7° to 15°. In some embodiments, the planar portions of top side 112, 112′ and bottom side 114, 114′ are configured to abut, at least partially, against opposing surfaces of the facet joint. In some embodiments, the planar portions of top side 112, 112′ and bottom side 114, 114′ extend the entire width W of body 102 (e.g., between lateral sides 108, 110). In some embodiments, the planar portions of top side 112, 112′ and bottom side 114, 114′ make up the majority of the area of top side 112, 112′ and bottom side 114, 114′. In some embodiments, the planar portions of top side 112, 112′ and bottom side 114, 114′ are continuous and do not include any holes or openings. In some embodiments, the planar portions of top side 112, 112′ and bottom side 114, 114′ are substantially flat and do not include any projections or protrusions.

In some embodiments, body 102 is constructed from a solid biocompatible material that is sufficiently rigid to withstand the compressive load within the facet joint without changing shape. In some embodiments, body 102 is made from a metal or metal alloy, for example, but not limited to, titanium, titanium alloys (e.g., Ti-6Al-4V), cobalt-chromium alloys, tantalum, or stainless steel. In other embodiments, body 102 is constructed from a ceramic material, for example, but not limited to, aluminum oxides or calcium phosphates. In other embodiments, body 102 may be made from or include a biocompatible thermoplastic material, for example, polyether ether ketone (PEEK). Composite materials that combine two or more different materials (e.g., metal/thermoplastic or metal/ceramic, etc.) may also be used to construct body 102, according to some embodiments. In some embodiments, body 102 is not composed of a bioabsorbable and/or biodegradable material.

Body 102 may be constructed via a process selected from additive manufacturing (e.g., 3D printing), machining (e.g., CNC machining), molding, casting, sintering, or other suitable manufacturing processes. In some embodiments, any external edges and/or corners of body 102 may be slightly rounded. In some embodiments, having rounded edges and/or corners may reduce the risk of gouging or fracturing bony portions of the facet joint during insertion of implant 100. In some embodiments, one or more surfaces of body 102 may be further processed by a surface treatment. The surface treatment may be applied to some or all of the external surfaces of body 102. In some embodiments, the surface treatment may be applied to body 102 in order to, for example, increase the contact area of implant 100 with the bone cement and/or bone tissue of the facet joint. In some embodiments, the surface treatment may be applied to body 102 to adjust or improve chemical and/or physical characteristics of implant 100, for example, corrosion resistance, wear resistance, hardness, etc. The surface treatment can include, for example, polishing, acid-etching, grit blasting, laser-etching, texturing, anodizing, oxidizing, plasma spraying, and/or chemical coating. In some embodiments, a surface treatment is applied to one or more portions of body 102 that is configured to promote or encourage bony ongrowth and/or ingrowth into implant 100. In some embodiments, implant 100 may have a porous surface. In some embodiments, the surface treatment includes applying a micro- or nano-scale texture onto a surface of body 102. For example, in some embodiments, micro- or nano-sized grooves or depressions may be formed on a surface of body 102 (e.g., by laser etching). In some embodiments, texturing on body 102 may include features that have dimensions (e.g., height, width, length, spacing, diameter, etc.) that are less than 100 μm. In some embodiments, the texturing may include features that have dimensions that are less than 50 μm. In some embodiments, the texturing may include features that have dimensions that are less than 10 μm. In some embodiments, the texturing may include features that have dimensions that are less than 5 μm. In some embodiments, the texturing may include features that have dimensions that are less than 1 μm.

Referring again to FIGS. 3A-3E, in some embodiments, body 102 includes a first channel 118 that extends through the length of body 102 from a proximal opening 116 at proximal end 106 to a distal opening 114 at distal end 104. As will be described further herein, in some embodiments first channel 118 is configured to provide a passage through which bone cement may be introduced after implant 100 has been inserted into the facet joint. In some embodiments, first channel 118 may be aligned with and disposed about an axis A1 that is a central axis of body 102. In some embodiments, axis A1 is located midway between lateral sides 108 and 110. In some embodiments axis A1 is located midway between top side 112 and bottom side 114. In some embodiments, body 102 may include at least one plane of symmetry that intersects with axis A1. In some embodiments, body 102 may include at least two planes of symmetry that intersect with axis A1. In some embodiments, body 102 includes at most or only two planes of symmetry. The two planes of symmetry are perpendicular to each other according to some embodiments. In some embodiments, the two planes of symmetry are perpendicular to each other and intersect at axis A1.

In some embodiments, first channel 118 includes a circular cross-sectional shape and may have a generally smooth interior surface (e.g., unthreaded). In some embodiments, first channel 118, proximal opening 116, and distal opening 114 have a constant diameter selected to be any value from 1.75 mm to 3.5 mm. In some embodiments, the diameter is selected to be 0.25 mm to 0.5 mm less than height H of body 102.

In some embodiments, body 102 includes at least a second channel 120 that extends through the length of body 102 from a proximal opening 124 at proximal end 106 to a distal opening 122 at distal end 104. Second channel 120 may be generally parallel to first channel 118 according to some embodiments, and may be positioned at a location between first channel 118 and one of lateral sides 108 or 110. In some embodiments, second channel 120 does not intersect or communicate with first channel 118. In the illustrated embodiment, second channel 120 is positioned between first channel 118 and lateral side 108. In some embodiments, second channel 120 has the same cross-sectional shape as first channel 118. In other embodiments, second channel 120 has a cross-sectional shape that is different from first channel 118. In some embodiments, second channel 120 includes a non-circular cross-sectional shape. For example, in some embodiments, second channel 120 may have a rectangular cross-sectional shape as illustrated. In some such embodiments, each of proximal opening 124 and distal opening 122 may also be rectangular and have dimensions, for example, of 2 mm width×1.5-3.5 mm height. Other shapes are also possible for second channel 120, proximal opening 124, and distal opening 122.

In some embodiments, body 102 includes a third channel 126 that extends through the length of body 102 from a proximal opening 130 at proximal end 106 to a distal opening 128 at distal end 104. Third channel 126 may be generally parallel to first channel 118 and/or second channel 120 according to some embodiments and may be arranged symmetrically with respect to second channel 120 about axis A1. In other embodiments, third channel 126 is not necessarily parallel to first channel 118 and/or second channel 120. In some embodiments, second channel 120 and/or third channel 126 may each have a generally smooth interior surface (e.g., unthreaded). In the illustrated embodiment, third channel 126 is positioned between first channel 118 and lateral side 110. In some embodiments, third channel 120 does not intersect or communicate with first channel 118 and/or second channel 120. In some embodiments, third channel 126 has the same cross-sectional shape as first channel 118 and/or second channel 120. In other embodiments, third channel 126 has a cross-sectional shape that is different from first channel 118 and/or second channel 120. In some embodiments, third channel 126 includes a non-circular cross-sectional shape. For example, in some embodiments, third channel 126 may also have a rectangular cross-sectional shape as illustrated. In some such embodiments, each of proximal opening 130 and distal opening 128 may also be rectangular and have dimensions, for example, of 2 mm width×1.5-3.5 mm height. Other shapes are also possible for third channel 126, proximal opening 130, and distal opening 128.

In some embodiments, second channel 120 and/or third channel 126 are configured to serve as egress channels for the bone cement. In some embodiments, providing such egress channels may help prevent over-pressurization of the facet joint with bone cement. In some embodiments, as will be described further herein, after implant 100 is inserted into a facet joint, bone cement is introduced through proximal opening 116 of body 102, passes through first channel 118, and at least a portion of the bone cement exits first channel 118 at distal opening 114. An amount of the bone cement that exists distal opening 114 may fill spaces between implant 100 and the surfaces of the facet joint. In some embodiments, excess bone cement may be channeled from the facet joint through second channel 120 and/or third channel 126. In some embodiments, bone cement reenters implant 100 through distal openings 122 and/or 128, flows through second channel 120 and/or third channel 126, and exits through proximal openings 124 and/or 130.

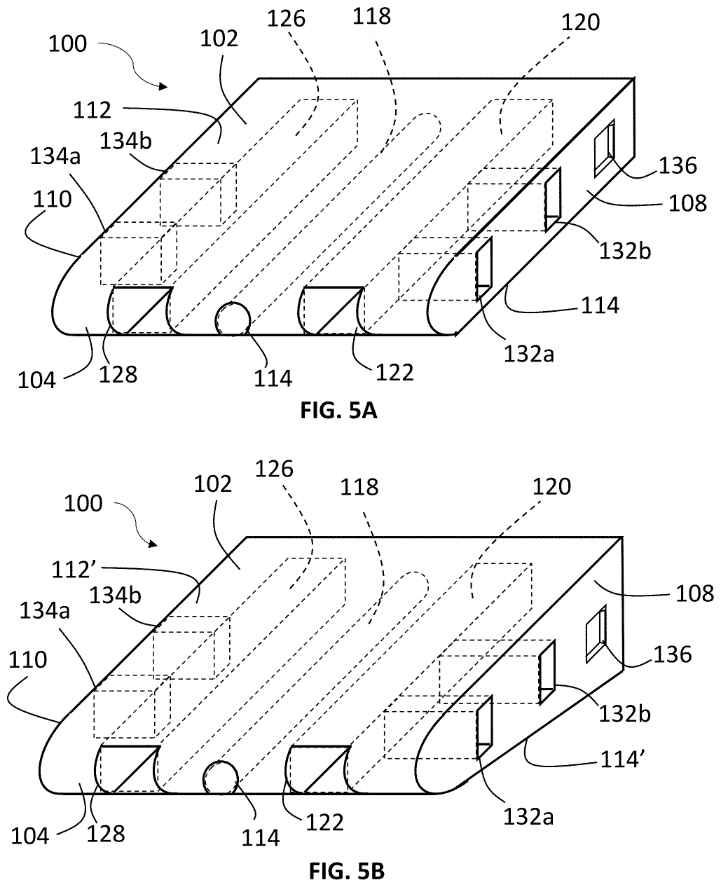

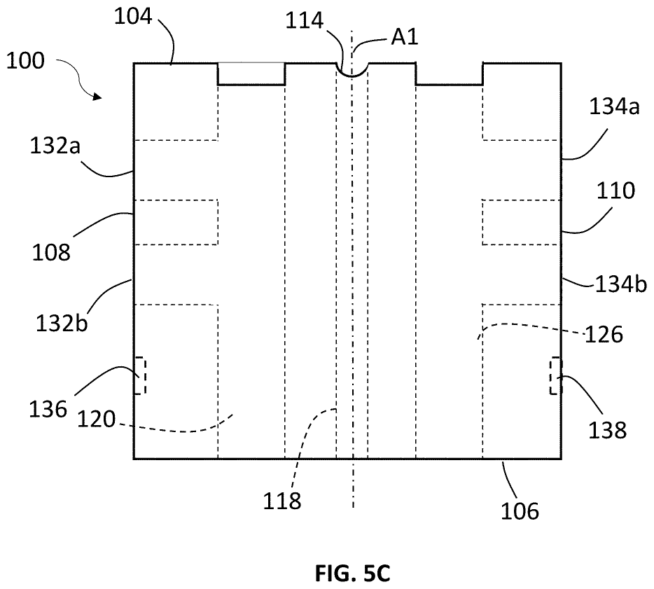

In some embodiments, body 102 includes one or more lateral openings in lateral side 108 and/or lateral side 110. In some embodiments, the one or more lateral openings are in fluid communication with either second channel 120 or third channel 126. In some such embodiments, bone cement flowing through second channel 120 and/or third channel 126 may exit through the one or more lateral openings rather than proximal openings 124 and/or 130. In some embodiments, the one or more lateral openings may help distribute bone cement around the lateral sides of implant 100 within the facet joint. FIGS. 5A-5C show example embodiments of implant 100 having one or more lateral openings 132a, 132b in lateral side 108 and one or more lateral openings 134a, 134b in lateral side 110. In some embodiments, lateral openings 132a, 132b connect to and are in fluid communication with second channel 120. In some embodiments, lateral openings 134a, 134b connect to and are in fluid communication with third channel 126. While two lateral openings are shown on each of lateral sides 108 and 110 in the illustrated embodiments, implant 100 may include fewer or more lateral openings according to other embodiments. Furthermore, while lateral openings 132a, 132b, 134a, and 134b are shown as having similar shapes and sizes, they may have different configurations in other embodiments.

In some embodiments, the cross-sectional areas of first, second, and/or third channels 118, 120, 126 may be selected based on the fluid properties of the bone cement (e.g., viscosity) such that, for example, the bone cement can flow readily through the channels and openings. For example, channels with larger cross-sectional area may be selected for use with bone cements having higher viscosity. In some embodiments, the cross-sectional areas of first, second, and/or third channels 118, 120, 126 may be sized such that the bone cement may flow through the channels without causing a reorientation of implant 100 during introduction of the bone cement material and/or without the bone cement becoming clogged in the channels or the openings connected thereto.

In some embodiments, implant 100 includes one or more features that are positioned and configured to engage with a tool to allow a user to manipulate implant 100. In some embodiments, the one or more features may be discrete surface features. The one or more features may include, for example, a recess, groove, notch, indentation, protrusion, lip, flange, or the like. In some embodiments, the one or more features are positioned at predetermined positions on one or more external surfaces of body 102. For example, the one or more features for engaging a tool may be located on top side 112, bottom side 114, distal end 104, proximal end 106, and/or lateral sides 108, 110. As shown in the illustrated embodiments of FIGS. 3A-5C, implant 100 may include indents 136 and 138 that are configured to engage with a tool. In some embodiments, indent 136 is located on lateral side 108, and indent 138 is located on lateral side 110. In some embodiments, indent 136 may be symmetrically positioned with respect to indent 136 about axis A1. In some embodiments, indents 136 and 138 are located closer to proximal end 106 than distal end 104. In some embodiments, indent 136 is located on lateral side 108 between proximal end 106 and the one or more lateral openings 132a, 132b. In some embodiments, indent 138 is located on lateral side 108 between proximal end 106 and the one or more lateral openings 134a, 134b. While indents 136, 138 are shown as having a square or rectangular shape, indents 136, 138 are not necessarily limited to this configuration and may have different sizes or other shapes (e.g., circular). In alternative embodiments (not shown), indents 136, 138 may be located on top side 112 and bottom side 114 rather than lateral sides 108, 110.

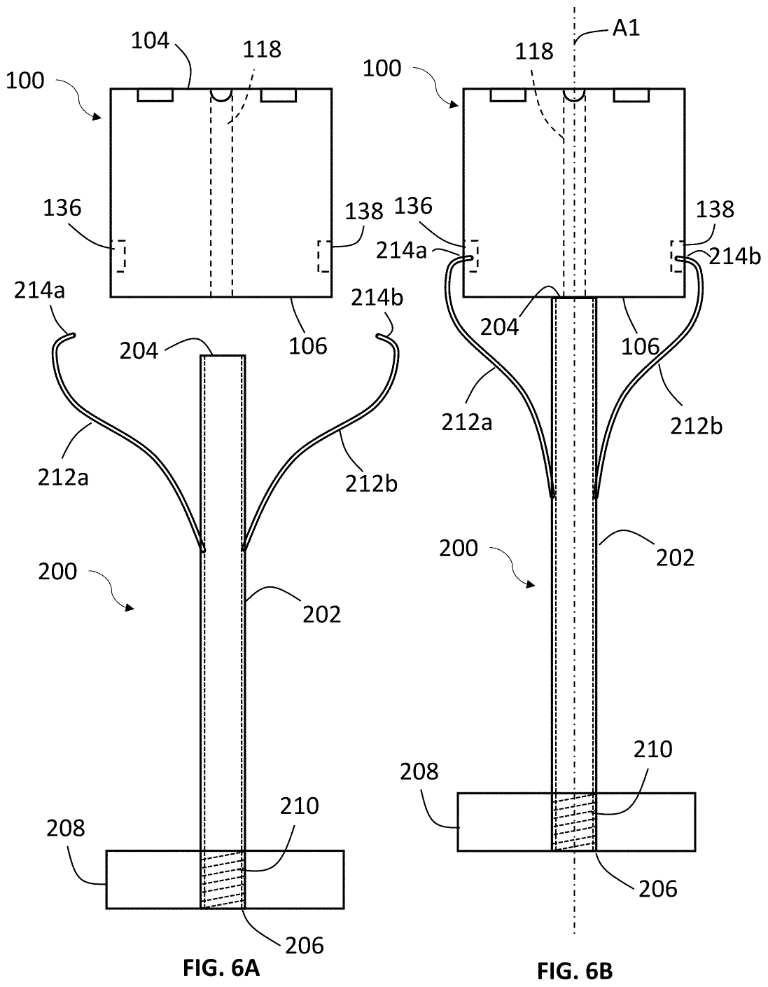

FIGS. 6A and 6B illustrate an example inserter tool 200 for engaging with implant 100. Inserter tool 200, according to some embodiments, is configured to hold implant 100 and be used for inserting implant 100 into a facet joint. In further embodiments, as will be described herein, inserter tool 200 may be used in combination with other tools for carrying out methods according to the present disclosure. In some embodiments, inserter tool 200 includes a hollow shaft 202 that is open at distal end 204 and proximal end 206. In some embodiments, shaft 202 has a circular cross-sectional profile. Shaft 202, in some embodiments, includes is made from a tube of rigid material, for example, metal or metal alloy (e.g., stainless steel). In some embodiments, shaft 202 includes a threaded section 210 that may be located at or proximate proximal end 206. Threaded section 210 of shaft 202, in some embodiments, includes an internal screw thread that is configured to mate with external thread of other tools. In some embodiments, inserter tool 200 may further include a handle or flange 208 disposed, at least partially, around a portion of shaft 202. In some embodiments, handle or flange 208 is disposed around the proximal end 206 of shaft 202. In some embodiments, handle or flange 208 is disposed around threaded section 210 of shaft 202.

In some embodiments, inserter tool 200 includes a grasping or clamping mechanism for engaging with implant 100. In the illustrated embodiments, inserter tool 200 includes a pair of arms 212a, 212b for holding onto implant 100. In some embodiments, arms 212a, 212b may be attached to and extend from shaft 202. In some embodiments, arms 212a, 212b are arranged on opposite sides of shaft 202 (e.g., diametrically opposed). In some embodiments, arms 212a, 212b are configured to move or pivot relative to shaft 202 and/or each other. In some embodiments, arms 212a, 212b can transition between a disengaged configuration shown in FIG. 6A and an engaged configuration shown in FIG. 6B. In some embodiments, arms 212a, 212b are configured to hold implant 100 in the engaged configuration. In some embodiments, arms 212a, 212b are configured to pivot towards each other when transitioning from the disengaged configuration to the engaged configuration, and arms 212a, 212b are configured to pivot away from each other when transitioning from the engaged configuration to the disengaged configuration. In some embodiments, inserter tool 200 may include one or more biasing elements (e.g., springs) that are configured to bias arms 212a, 212b toward the engaged configuration. In some embodiments, arms 212a, 212b include distal ends 214a, 214b that may be received within indents 136, 138 to hold implant 100 when inserter tool 200 is in the engaged configuration. Distal ends 214a, 214b may, for example, be hooked, curved, or angled in an inward direction (toward the axis of shaft 202) relative to the rest of arms 212a, 212b. Arms 212a, 212b may thus have a generally S-shape or question mark shape (“?”) according to some such embodiments. In some embodiments, when in the engaged configuration, distal end 204 of shaft 202 abuts against proximal end 106 of implant 100. In further embodiments, shaft 202 may be disposed around axis A1 of implant 100 such that the hollow of shaft 202 is coaxial with first channel 118 of implant 100. In some embodiments, shaft 202 has an inner diameter (the diameter of the hollow space within shaft 202), that is larger than the diameter of hole 118. In some embodiments, shaft 202 has an outer diameter that is smaller than height H of implant 100.

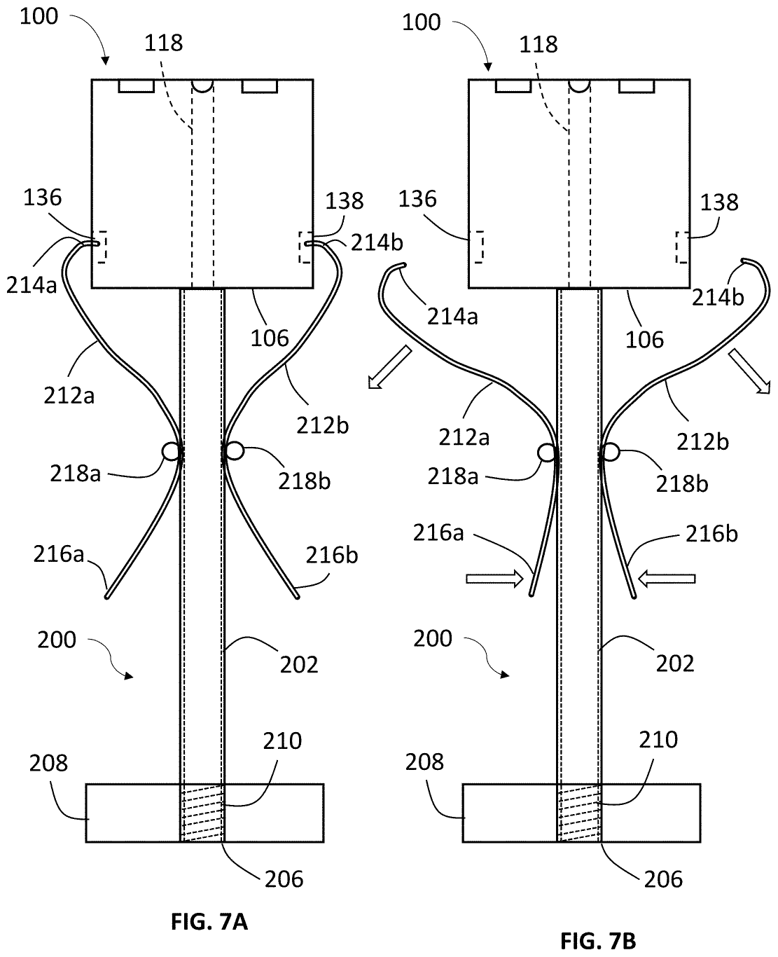

FIGS. 7A and 7B illustrate a particular embodiment of inserter tool 200 wherein arms 212a, 212b are configured to pivot about fulcrums 218a, 218b, respectively. In some embodiments, arms 212a, 212b further include proximal ends 216a, 216b, respectively, with fulcrums 218a, 218b being positioned against arms 212a, 212b at a location between distal ends 214a, 214b and proximal ends 216a, 216b. In some embodiments, arms 212a, 212b may be tensioned or biased toward the engaged configuration (FIG. 7A). In some such embodiments, arms 212a, 212b are shaped such that pressing arms 212a, 212b at proximal ends 216a, 216b towards shaft 202 causes distal ends 214a, 214b to pivot away from and disengage from implant 100, as depicted by the arrows in FIG. 7B. In some embodiments, arms 212a, 212b may have, for example, an S-shaped curve or serpentine curve.

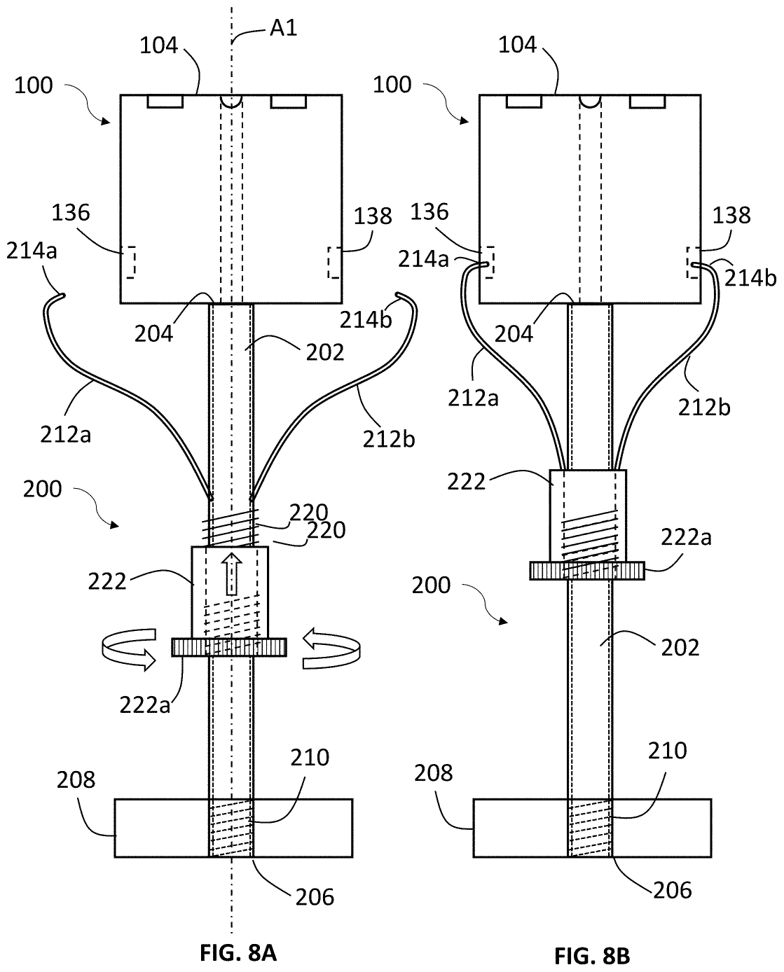

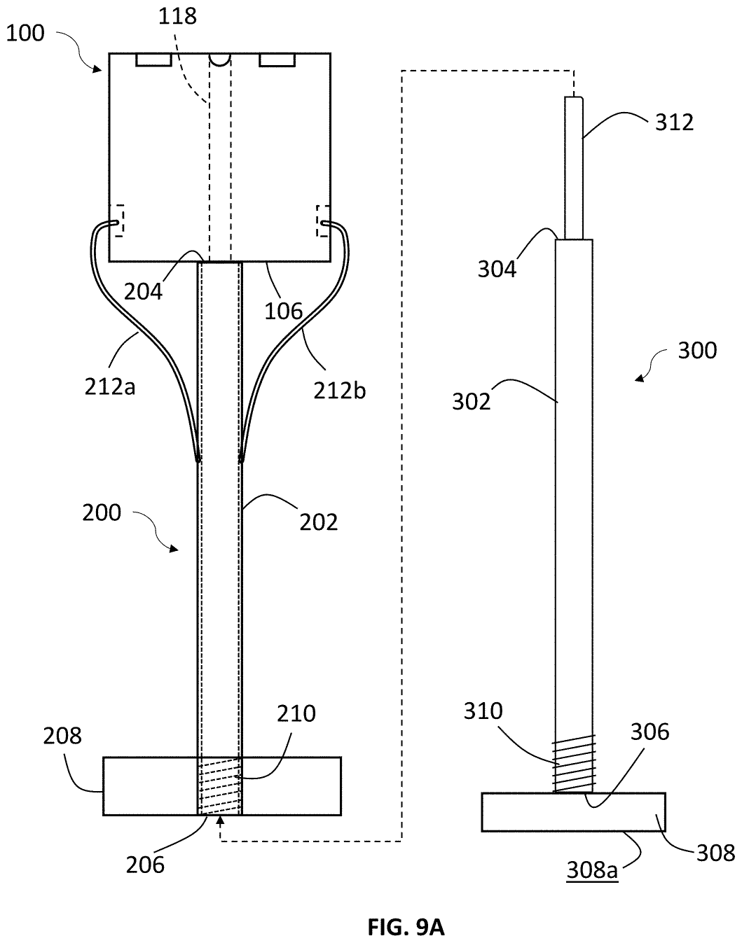

FIGS. 8A and 8B illustrate an alternative embodiment of inserter tool 200 including a collar 222 that is configured to move with respect to shaft 202. In some embodiments, collar 222 is configured to translate axially along shaft 202 (e.g., in a distal/proximal direction). In some embodiments, collar 222 is configured to rotate about shaft 202. In some embodiments, collar 222 is configured to rotate about axis A1. In some such embodiments, shaft 202 includes an externally threaded section 220, which may be located at or proximate to where arms 212a, 212b are attached to shaft 202. Collar 222, in some embodiments, is disposed around shaft 202 and includes internal threads that are configured to engage with the external threads of externally threaded section 220. In some embodiments, rotating collar 222 around shaft 202 in a first rotational direction causes collar 222 to translate in a distal direction (e.g., toward distal end 204) along externally threaded section 220. Rotating collar 222 in a second, opposite rotational direction causes collar 222 to translate in a proximal direction (e.g., toward proximal end 206). Collar 222 may be configured to be manually rotated by the user according to some embodiments. In some embodiments, collar 222 can include a wheel 222a configured to be rotated by hand to facilitate rotation of collar 222. Wheel 222a, for example, may be a flange projecting from collar 222 and may have a textured or knurled surface. In some embodiments, as collar 222 translates toward distal end 204, collar 222 is configured to slide over and compress arms 212a, 212b towards shaft 202 and each other, allowing inserter tool 200 to transition to the engaged configuration, as depicted in FIG. 8B. In some embodiments, causing collar 222 to translate toward proximal end 206 of shaft 202 allows arms 212a, 212b to pivot away from each other and transition to a disengaged configuration.

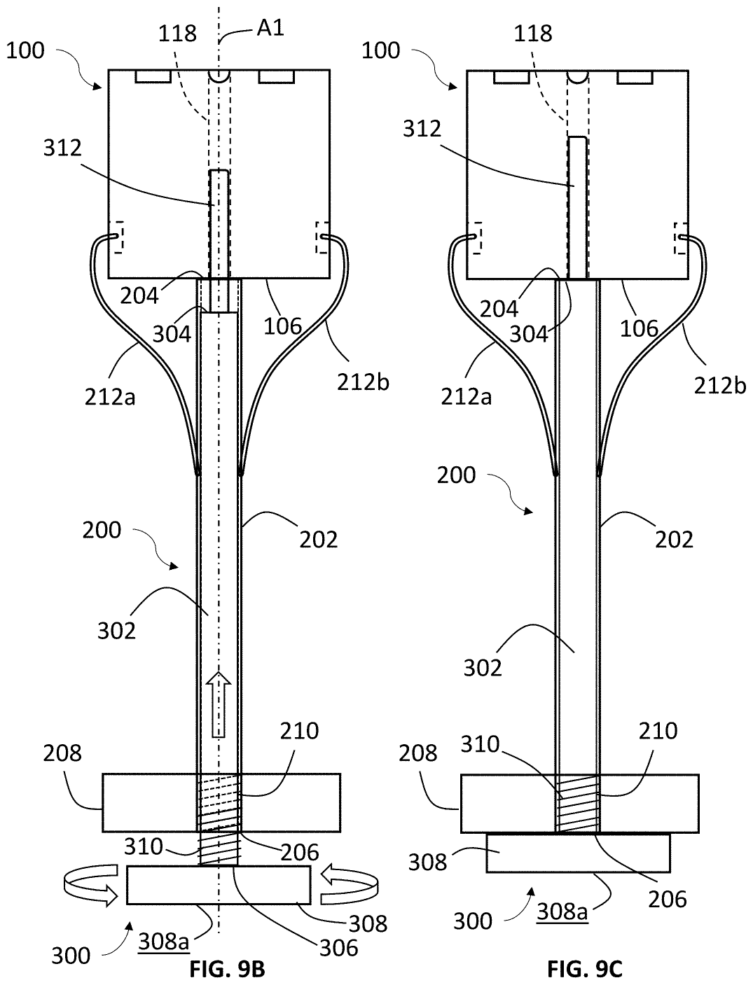

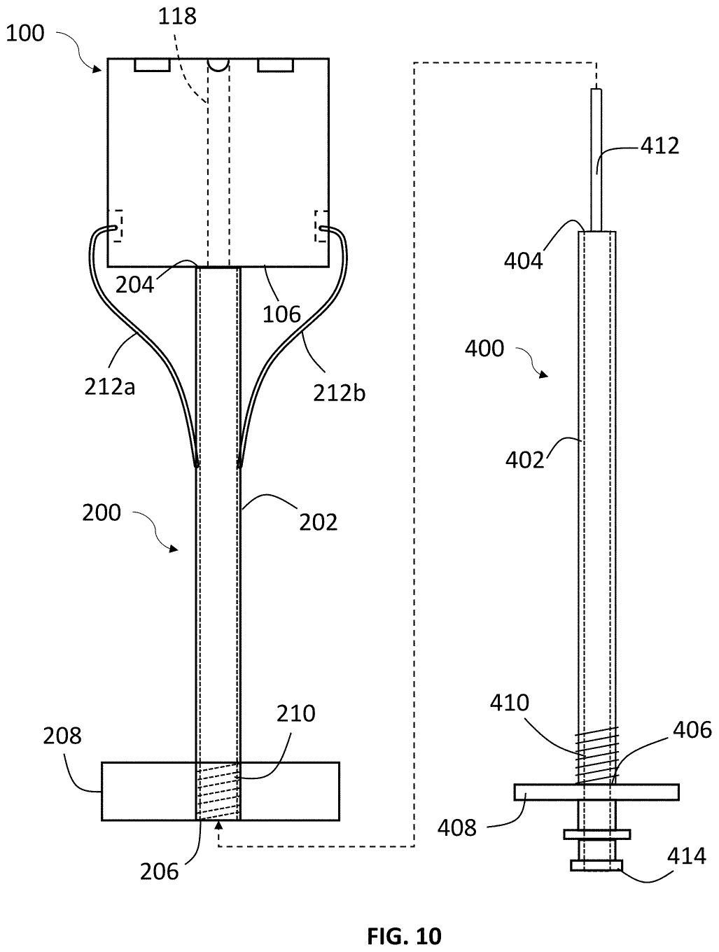

In some embodiments, inserter tool 200 is configured to receive one or more additional tools. The one or more additional tools may be inserted through open proximal end 206 of shaft 202 in some embodiments. With reference now to FIGS. 9A-9C, inserter tool 200 in some embodiments is configured to receive an impaction tool 300. In some embodiments, impaction tool 300 provides an impact surface for receiving a force to drive implant 100 into the facet joint during implantation. Impaction tool 300, in some embodiments, is further configured to help stabilize implant 100 and inserter tool 200 during implantation of implant 100 into the facet joint. In some embodiments, impaction tool 300 includes a shaft 302 that is sized to be inserted into shaft 202 of inserter tool 200. Shaft 302 may be solid and rigid and made from materials such as metal or metal alloys (e.g., stainless steel) according to some embodiments. In some embodiments, impaction tool 300 is sized and configured to rotate (e.g., about axis A1) within shaft 202. In some embodiments, shaft 302 includes a distal end 304 and a proximal end 306. A distance (e.g., length) between distal end 304 and proximal end 306 of shaft 302, in some embodiments, is equal to the distance between distal end 204 and proximal end 206 of shaft 202 of inserter tool 200. In some embodiments, shaft 302 is cylindrical and includes a diameter that is smaller than the inner diameter of shaft 202. In some embodiments, the diameter of shaft 302 may be larger than the diameter of first channel 118 of implant 100.

In some embodiments, shaft 302 includes an externally threaded section 310. Threaded section 310, in some embodiments is located at or proximate proximal end 306 of shaft 302. Threaded section 310 in some embodiments includes an external thread that is sized and positioned to couple with the internal thread of internally threaded section 210 of shaft 202 when impaction tool 300 is received within inserter tool 200, as particularly shown in FIG. 9B. In some such embodiments, impaction tool 300 may be slid into shaft 202 via open proximal end 206 until externally threaded section 310 engages with internally threaded section 210, at which point impaction tool 300 must be rotated in a first rotational direction relative to shaft 202 to advance impaction tool 300 in the axial direction. In some embodiments, when impaction tool 300 is fully coupled with inserter tool 200 (e.g., as shown in FIG. 9C), distal end 304 of shaft 302 is flush with distal end 204 of shaft 202. In some embodiments, distal end 304 of shaft 302 is configured to abut against proximal end 106 of implant 100 when impaction tool 300 is fully coupled with inserter tool 200.

In some embodiments, impaction tool 300 further includes a head 308 that is fixed to shaft 302 at proximal end 306. Head 308 includes an end surface 308a is configured to receive a force to drive implant 100 into the facet joint during implantation. For example, a mallet, hammer, or other impact tool may be used to apply a driving force against end surface 308a when impaction tool 300 is engaged with inserter tool 200 to drive implant 100 into the facet joint. End surface 308a may be a flat surface, in some embodiments. In some embodiments, head 308 is positioned to abut against handle or flange 208 of inserter tool 200 when impaction tool 300 is fully coupled with inserter tool 200.

In further embodiments, impaction tool 300 includes a tip portion 312 opposite head 308. In some embodiments, tip portion 312 extends from distal end 304 of shaft 302 and has a diameter that is smaller than the diameter of shaft 302. In some embodiments, tip portion 312 is sized to be inserted into first channel 118 of implant 100 (e.g., via proximal opening 116) when impaction tool 300 is inserted into inserter tool 200 and may have a cylindrical shape. In some embodiments, tip portion 312 assists in stabilizing implant 100 during implantation by restricting movement of implant 100 relative to inserter tool 200 and impaction tool 300. For example, in some embodiments, tip portion 312 inserted into first channel 118 may assist in keeping implant 100 axially aligned with impaction tool 300 and inserter tool 200 during implantation.

In some embodiments, after implant 100 has been positioned within the facet joint, impaction tool 300 may be withdrawn from inserter tool 200. In some embodiments, impaction tool 300 may be withdrawn by rotating impaction tool 300 relative to inserter tool 200 in a direction to unscrew impaction tool 300 from inserter tool 200 and disengage the external thread of threaded portion 310 from the internal thread of threaded portion 210. In some embodiments, after impaction tool 300 has been removed from inserter tool 200, a separate tool may be received by inserter tool 200 to facilitate the introduction of bone cement into the facet joint.

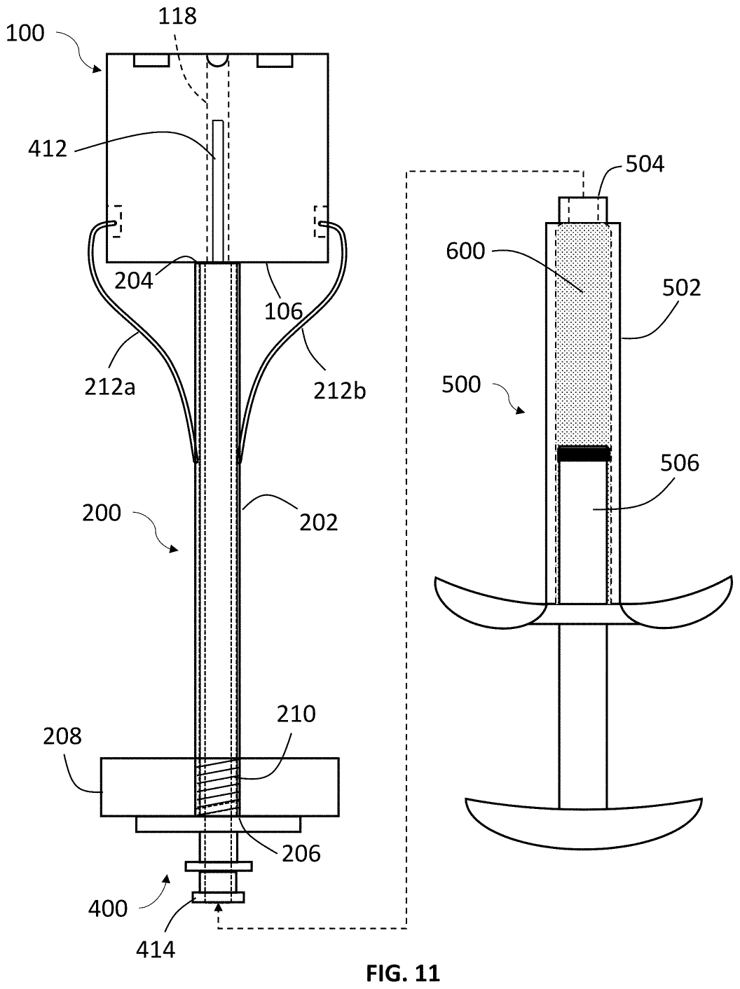

Referring now to FIGS. 10 and 11, there is a shown a cement introducer 400 that may be used in combination with inserter tool 200. In some embodiments, cement introducer 400 provides a channel for conducting bone cement from a bone cement source (e.g., syringe 500) to implant 100. In some embodiments, cement introducer 400 includes a hollow shaft 402 that is sized to be inserted into shaft 202 of inserter tool 200. In some embodiments, cement introducer 400 is sized and configured to rotate (e.g., about axis A1) within shaft 202. In some embodiments, shaft 302 includes a distal end 404 and a proximal end 406. A distance (e.g., length) between distal end 404 and proximal end 406 of shaft 402, in some embodiments, is equal to the distance between distal end 204 and proximal end 206 of shaft 202 of inserter tool 200. In some embodiments, shaft 402 is cylindrical and includes an outer diameter that is smaller than the inner diameter of shaft 202. In some embodiments, the outer diameter of shaft 402 may be larger than the diameter of first channel 118 of implant 100.

In some embodiments, shaft 402 includes an externally threaded section 410. Threaded section 410, in some embodiments is located at or proximate proximal end 406 of shaft 402. Threaded section 410 in some embodiments includes an external thread that is sized and positioned to couple with the internal thread of internally threaded section 210 of shaft 202 when cement introducer 400 is received within inserter tool 200. In some such embodiments, cement introducer 400 may be slid into shaft 202 via open proximal end 206 until externally threaded section 410 engages with internally threaded section 210, at which point cement introducer 400 must be rotated in a first rotational direction relative to shaft 202 to advance cement introducer 400 in the axial direction. In some embodiments, when cement introducer 400 is fully coupled with inserter tool 200 (e.g., as shown in FIG. 11), distal end 404 of shaft 402 is flush with distal end 204 of shaft 202. In some embodiments, distal end 404 of shaft 402 is configured to abut against proximal end 106 of implant 100 when cement introducer 400 is fully coupled with inserter tool 200.

In further embodiments, cement introducer 400 includes a hollow tip portion 412 that extends from distal end 404 of shaft 402 and has a diameter that is smaller than the diameter of shaft 402. In some embodiments, tip portion 412 is sized to be inserted into first channel 118 of implant 100 (e.g., via proximal opening 116) when cement introducer 400 is inserted into inserter tool 200. In some embodiments, hollow tip portion 412 is configured to conduct bone cement from shaft 402 into implant 100.

In some embodiments, cement introducer 400 includes a port 414 that extends from shaft 402 at proximal end 406. Port 414, in some embodiments, is configured to couple with a bone cement source (e.g., syringe 500) to receive bone cement therefrom. In some embodiments, port 414 includes a fitting that is configured to form a fluid-tight connection to the bone cement source. For example, in some embodiments port 414 may include a Luer lock fitting, threaded fitting, quick connect fitting, or other fitting. In some embodiments, cement introducer 400 further includes a flange 408 that is positioned to abut against handle or flange 208 of inserter tool 200 when cement introducer 400 is fully coupled with inserter tool 200. In some embodiments, flange 408 is axially positioned between proximal end 406 and port 414.

As illustrated in FIG. 11, the bone cement source may be, for example, a syringe 500 according to some embodiments. In general, syringe 500 may include a barrel 502 containing a bone cement material 600 and a plunger or piston 506 configured to expel bone cement material 600 out of open end 504. Bone cement material 600, according to some embodiments, may be in a fluid or flowable state (e.g., an uncured state) while contained in the bone cement source. Bone cement material 600 may include, for example, PMMA or other acrylic bone cements, CPCs, HBCs, CSCs, or combinations thereof (e.g., calcium phosphate/hydroxyapatite or PMMA/calcium phosphate composite materials, etc.). In some embodiments, bone cement material 600 may include any known bone cement that is useful for kyphoplasty or vertebroplasty. In some embodiments, the bone cement material 600 is or includes PMMA or other material that mimics the properties of PMMA. In some embodiments, bone cement material 600 is or includes one or more bone cement precursor materials, for example, one or more reactants that react to form the bone cement. In some embodiments, open end 504 is configured to couple to port 414 of cement introducer 400. In some embodiments, open end 504 is configured to directly couple to port 414 (e.g., via a Luer lock fitting or other fitting). In other embodiments, open end 504 may be indirectly coupled to port 414 via, for example, tubing that connects open end 504 to port 414. The bone cement source is not necessarily limited to a syringe, and other bone cement applicators and dispensers known in the art may be utilized with embodiments of the present disclosure.

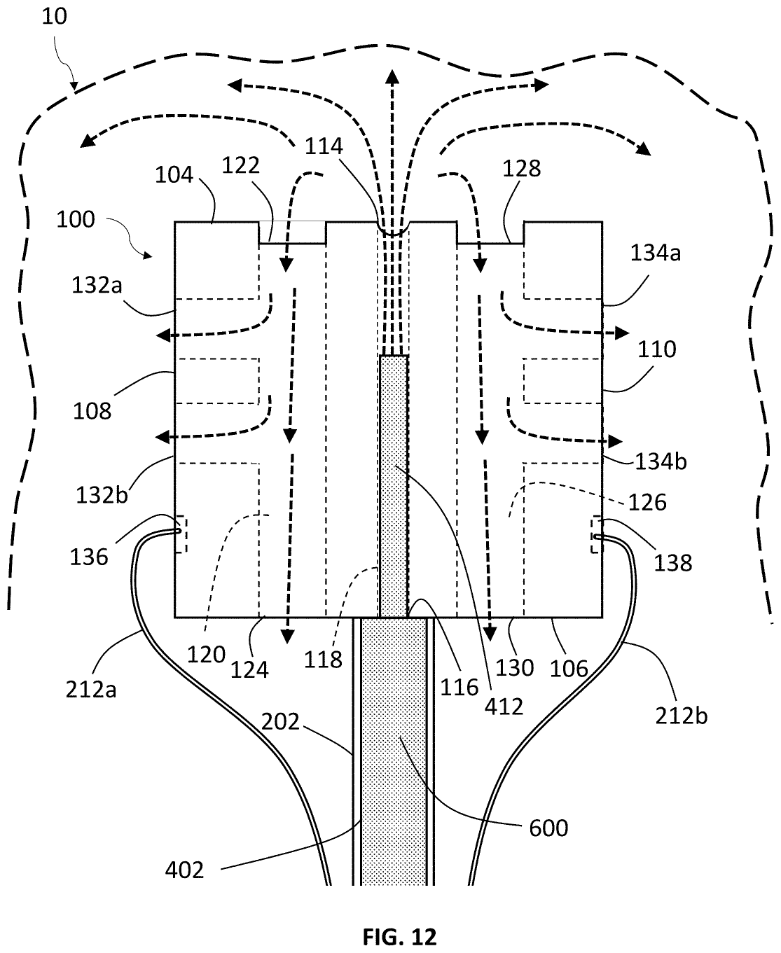

In some embodiments, after implant 100 is positioned within the facet joint, bone cement material is dispensed from the bone cement source and travels through port 414, shaft 402, and hollow tip portion 412 of cement introducer 400 into implant 100. FIG. 12 illustrates implant 100 positioned within facet joint 10 according to some such embodiments. Bone cement material 600, dispensed from the bone cement source, flows through shaft 402 and into hollow tip portion 412 located within first channel 118 of implant 100 where the bone cement material 600 is expelled. The expelled bone cement material 600 continues to flow through first channel 118 and exits implant 100 at distal opening 114 to fill, at least partially, a space of the facet joint surrounding implant 100. The flow of bone cement material 600, according to some embodiments, is depicted by the dashed arrows.

In some embodiments, at least a portion of the bone cement material 600 may flow back into implant 100 after exiting first channel 118. In some embodiments, bone cement material 600 may flow back into implant 100 via distal openings 122 and 128 of second and third channels 120 and 126, respectively. In some embodiments, second channel 120 and third channel 130 are configured to serve as egress channels for the bone cement material 600. In some embodiments, providing such egress channels may help prevent over-pressurization of the facet joint with the bone cement material 600. In some embodiments, excess bone cement material 600 that reenters implant 100 through distal openings 122 and/or 128 may flow through second channel 120 and/or third channel 126, and exit implant 100 through proximal openings 124 and/or 130. In some embodiments, where implant 100 includes one or more lateral openings 132a, 132b, 134a, 134b, bone cement flowing through second channel 120 and/or third channel 126 may exit implant 100 through the one or more lateral openings rather than proximal openings 124 and/or 130. In some embodiments, the one or more lateral openings 132a, 132b, 134a, 134b may help distribute bone cement around the lateral sides of implant 100 within the facet joint. In some embodiments, once bone cement material 600 is observed exiting second channel 120 and/or third channel 126, the introduction of the bone cement material 600 from the bone cement source may be ceased, and any bone cement material that has extruded out of the facet joint or implant 100 from proximal openings 124, 130 should be removed. In some embodiments, after the introduction of the bone cement material 600 has ceased, the cement introducer 400 and inserter tool 200 may be removed from implant 100 and the bone cement material 600 is allowed to cure and harden in the facet joint around implant 100.

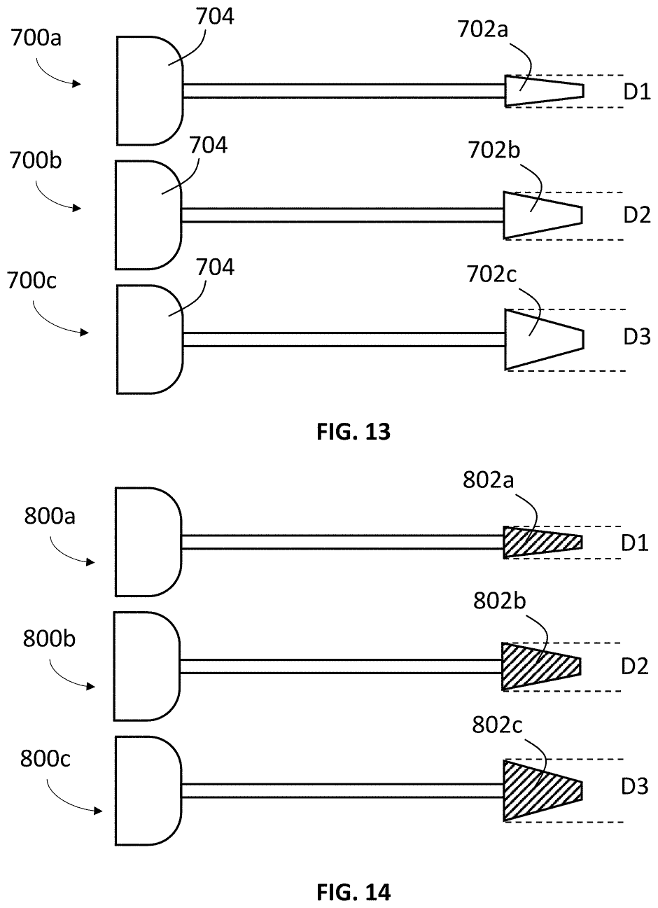

In some embodiments, the facet joint may require certain preparation prior to implantation of implant 100. In some embodiments, a dorsal aspect of the facet joint capsule may be removed in order to provide an opening to the facet joint. In some such embodiments, electrocautery may be used to remove the dorsal aspect of the facet joint capsule. In further embodiments, once the opening of the facet joint is exposed, one or more trial gauges of predetermined size may be introduced into the facet joint in order to dilate the opening of the facet joint and to determine the appropriate height of implant 100 to be inserted. FIG. 13 illustrates a plurality of trial gauges 700a, 700b, 700c that may be provided, each having a tip 702a, 702b, 702c configured to be inserted into the opening of the facet joint. Each of tips 702a, 702b, 702c has a different predetermined height D1, D2, D3 and may be tapered (e.g., 7°-15°). D1, D2, D3 may range in size from 1 mm to 4 mm in some embodiments. For example, D1 may be 2 mm, D2 may be 3 mm, and D3 may be 4 mm. While three trial gauges are shown for simplicity, additional trial gauges of additional tip sizes may also be included. Each trial gauge 700a, 700b, 700c may further include a handle 704 connected to the tip 702a, 702b, 702c. In use, according to some embodiments, progressively larger trial gauges are inserted into the facet joint until an appropriate fit is achieved by the tip. In some embodiments, progressively larger trial gauges may be individually inserted into the facet joint until a trial gauge that provides a firm fit within the facet joint is found. In some embodiments, an implant 100 having a height H equal to the tip height of the final trial gauge found to fit within the facet joint may be selected for implantation. For example, in some embodiments, if a trial gauge with a tip height of 4 mm was found to fit the facet joint appropriately, an implant 100 having a height H of 4 mm may be chosen for implantation into the facet joint.

In some embodiments, surfaces of the facet joint may be rasped prior to inserting implant 100. In some embodiments, a rasp is used to remove cartilage and decorticate the facet joint surfaces. FIG. 14 illustrates a plurality of rasps 800a, 800b, 800c that may be provided, each having a textured tip 802a, 802b, 802c configured to be inserted into the facet joint and rasp the surfaces of the facet joint. In some embodiments, each of textured tips 802a, 802b, 802c has a different predetermined height D1, D2, D3 that are selected to equal heights D1, D2, D3 used for trial gauges 700a, 700b, 700c, respectively. In some embodiments, a rasp having a height equal to the height of the final trial gauge found to fit within the facet joint may be selected for rasping the facet joint. For example, in some embodiments, if a trial gauge with a tip height of 4 mm is found to fit the facet joint appropriately, a rasp having a tip height of 4 mm may be chosen for rasping surfaces of facet joint.

In some embodiments, two or more components described herein may be assembled as a kit. In some embodiments, a kit according to the present disclosure may include at least one implant 100 and at least one of the tools as described herein (e.g., inserter tool 200, impaction tool 300, cement introducer 400, trial gauges 700a-700c, or rasps 800a-800c). In some embodiments, a kit may include at least one implant 100 and inserter tool 200. In some embodiments, a kit may include at least one implant 100, inserter tool 200, and impaction tool 300. In some embodiments, a kit may include at least one implant 100, inserter tool 200, impaction tool 300, and cement introducer 400. In some embodiments, a kit may include at least one implant 100, inserter tool 200, and cement introducer 400. In some embodiments, kits according to the present disclosure may include a plurality of implants 100. In some embodiments, the plurality of implants 100 may have the same or different shapes and/or sizes. In further embodiments, a kit may include a plurality of trial gauges 700a-700c and rasps 800a-800c having different sizes. In yet further embodiments, a kit may include a bone cement source, for example, syringe 500 or other bone cement dispenser. Two or more components of a kit may be packaged together according to some embodiments. In some embodiments, implant 100 and any of the tools described herein (e.g., inserter tool 200, impaction tool 300, cement introducer 400, trial gauges 700a-700c, or rasps 800a-800c) are sterilized prior to packaging. In some embodiments, implant 100 and any of the tools described herein (e.g., inserter tool 200, impaction tool 300, cement introducer 400, trial gauges 700a-700c, or rasps 800a-800c) are sterilized prior to use.

It should be understood that various changes, substitutions, and alterations can be made herein without departing from the spirit and scope of the invention as defined by the appended claims. It should also be apparent that individual elements identified herein as belonging to a particular embodiment may be included in other embodiments of the invention. Moreover, the scope of the present application is not intended to be limited to the particular embodiments of the process, machine, manufacture, and composition of matter, means, methods and steps described in the specification. As one of ordinary skill in the art will readily appreciate from the disclosure herein, processes, machines, manufacture, composition of matter, means, methods, or steps, presently existing or later to be developed that perform substantially the same function or achieve substantially the same result as the corresponding embodiments described herein may be utilized according to the present invention.

What is claimed is:

1. An implant for insertion into a facet joint, the implant comprising:
a body sized to fit completely within the facet joint, the body comprising:
a proximal end;
a distal end opposite of the proximal end, the distal end comprising a tapered portion;
a first channel extending through the body from the proximal end to the distal end;
a second channel extending through the body from the proximal end to the distal end, the second channel being positioned between the first channel and a first lateral side of the body; and
a third channel extending through the body from the proximal end to the distal end, the third channel being positioned between the first channel and a second lateral side of the body;
a first tool engagement feature positioned on the first lateral side; and
a second tool engagement feature positioned on the second lateral side.

2. The implant of claim 1, wherein the body further comprises:
a top side having a first planar portion extending from the first lateral side to the second lateral side; and
a bottom side opposite the top side and having a second planar portion extending from the first lateral side to the second lateral side.

3. The implant of claim 2, wherein the first planar portion of the top side and the second planar portion of the bottom side are substantially parallel to each other.

4. The implant of claim 2, wherein the first planar portion of the top side is acutely angled with respect to the second planar portion of the bottom side.

5. The implant of claim 4, wherein the first planar portion of the top side is acutely angled with respect to the second planar portion of the bottom side by an angle selected from 7° to 15°.

6. The implant of claim 1, wherein the first tool engagement feature and the second tool engagement feature include one or more of an indent, recess, groove, slot, notch, protrusion, lip, flange, or the like sized and dimensioned to receive a portion of a tool.

7. The implant of claim 1, wherein the first channel is disposed about a central axis of the body.

8. The implant of claim 7, wherein the body includes at least one plane of symmetry that intersects with the central axis.

9. The implant of claim 8, wherein the body includes two perpendicular planes of symmetry that intersect at the central axis.

10. The implant of claim 1, wherein the first channel, the second channel, and the third channel are unthreaded.

11. The implant of claim 1, further comprising at least one opening on the first lateral side, the at least one opening on the first lateral side connecting to the second channel.

12. The implant of claim 1, further comprising at least one opening on the second lateral side, the at least one opening on the second lateral side connecting to the third channel.

13. The implant of claim 1, wherein the body is constructed from a metal or metal alloy.

14. The implant of claim 13, wherein the metal or metal alloy comprises titanium or a titanium alloy.

15. The implant of claim 1, wherein a width of the body between the first lateral side and the second lateral side is selected from a range of 5 mm to 8 mm, a length of the body between the proximal end and the distal end is selected from a range of 5 mm to 8 mm, and a height of the body at the proximal end is selected from a range of 2 mm to 4 mm.

16. A method for facet joint stabilization or fusion, the method comprising:
inserting the implant of claim 1 into a facet joint of a patient.

17. The method of claim 16, further comprising introducing a bone cement into the facet joint.

18. The method of claim 17, wherein the bone cement is introduced through the first channel of the body of the implant.

19. The method of claim 18, wherein a portion of the bone cement exits the first channel and at least partially fills a space of the facet joint surrounding the implant.

20. The method of claim 19, wherein a portion of the bone cement that exits the first channel is allowed to flow into the second channel and/or the third channel of the body of the implant.
